(19) United States
(12) Patent Application Publication
(10) Pub. No.: US 2014/0095203 A1
(43) Pub. Date: Apr. 3, 2014

(54) MEDICAL WORKFLOW DETERMINATION AND OPTIMIZATION

(71) Applicants: Vikram Anand, Downingtown, PA (US); Balaji Krishnapuram, King of Prussia, PA (US); Glenn Fung, Madison, WI (US); Shipeng Yu, Exton, PA (US); Faisal Farooq, Norristown, PA (US); Jonathan D. Emanuele, Phoenixville, PA (US)

(72) Inventors: Vikram Anand, Downingtown, PA (US); Balaji Krishnapuram, King of Prussia, PA (US); Glenn Fung, Madison, WI (US); Shipeng Yu, Exton, PA (US); Faisal Farooq, Norristown, PA (US); Jonathan D. Emanuele, Phoenixville, PA (US)

(73) Assignee: SIEMENS MEDICAL SOLUTIONS USA, INC., Malvern, PA (US)

(21) Appl. No.: 14/035,989

(22) Filed: Sep. 25, 2013

Related U.S. Application Data
(60) Provisional application No. 61/707,200, filed on Sep. 28, 2012.
(60) Provisional application No. 61/707,166, filed on Sep. 28, 2012.

(51) Int. Cl. G06Q 50/24; G06Q 10/06

(52) U.S. Cl. CPC G06Q 50/24; G06Q 10/0633
USPC 705/3

(57) ABSTRACT

Workflows for medical entities are determined and evaluated by determining a plurality of medical tasks based on an analysis of a plurality of electronic medical records of a medical entity. A workflow of the medical entity is determined based on a sequence of medical tasks, the sequence determined based on the analysis of the plurality of electronic medical records, and an evaluation of the workflow is performed based on a predefined criterion.

### RELATED APPLICATIONS

The present patent document claims the benefit of the filing date under 35 U.S.C. §119(e) of Provisional U.S. Patent Application Ser. No. 61/707,200, filed Sep. 28, 2012, and U.S. Patent Application Ser. No. 61/707,166, filed Sep. 28, 2012 which are hereby incorporated by reference.

### BACKGROUND

The present embodiments relate to medical workflow determination. Specifically, the present embodiments relate to automatic medical workflow determination and optimization using medical entity data.

Medical facilities and medical entities face challenges in improving the quality of care for patients, as well as reducing costs and increasing revenue. Efficient and effective medical care process, or workflow, design may aid in the pursuit of these goals by increasing process stability, repeatability, and effectiveness.

A medical workflow is a set of tasks generally having a designated order of performance. Typically, the set of tasks is designed to accomplish an objective. Some of the acts may be clinical in nature, and as such involve the acquisition of data for further diagnosis and analysis. Other acts may involve treatments, designated for treating medical conditions. The collection of acts that make up a workflow may be assembled for the diagnosis and/or treatment of certain medical conditions. A medical workflow may be formally or informally defined in a medical entity.

Significant amounts of information relating to the operation of medical entities may now be stored electronically. Electronic databases and logs of activity for the medical equipment of a medical entity are stored electronically. The schedules and activities of medical practitioners also may be stored electronically. Also, Electronic Medical Records (EMR) have become a standard storage technique for medical and health records for patients of medical practitioners and medical entities. EMRs contain a considerable amount of medical data for specific patients, from various sources and in various formats. Collections of EMRs for medical facilities provide medical records and history for most, if not all, patients in a medical entity.

### BRIEF SUMMARY

By way of introduction, the preferred embodiments described below include methods, computer readable media, and systems for workflow determination and/or optimization. Medical workflows may be determined from electronically stored records for a medical entity. The determined workflows may be representative of actual procedures or processes of the medical entity. The determined workflows may be compared to standards, or evaluated based on certain criteria, to identify potential improvements to the existing determined workflows.

In a first aspect, a method is provided for entity workflow evaluation. A plurality of medical tasks are determined based on an analysis of a plurality of electronic medical records of a medical entity. A workflow of the medical entity is determined based on a sequence of medical tasks, the sequence determined based on the analysis of the plurality of electronic medical records. An evaluation is performed of the workflow based on a predefined criterion.

In a second aspect, a non-transitory computer readable storage medium has stored therein data representing instructions executable by a programmed processor for evaluating workflows comprising medical tasks of a medical entity. A workflow is detected from an electronic medical record for a patient of a medical entity. A comparison is performed of the workflow to an established workflow. An anomalous medical task of the workflow is determined based on the comparison.

In a third aspect, a system is provided for medical entity workflow evaluation. A memory is operable to store data for a plurality of patients of a medical entity. A processor is configured to determine a plurality of medical tasks based on an analysis of the plurality of electronic medical records of a medical entity, determine a workflow of the medical entity based on a sequence of the medical tasks, the sequence determined based on the analysis of the plurality of electronic medical records, perform an evaluation of the workflow based on predefined criteria.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of an embodiment of a method for medical entity workflow evaluation;

FIG. 2 is a flow chart diagram of an embodiment of a method for evaluating workflows comprising medical tasks of a medical entity;

FIG. 3 is a block diagram of one embodiment of a system for medical entity workflow evaluation; and

FIG. 4 is a representation of an electronic medical record.

### DETAILED DESCRIPTION OF THE DRAWINGS AND PRESENTLY PREFERRED EMBODIMENTS

Medical workflows for a medical entity may be determined using EMRs of patients of the medical facility. The EMRs may represent a collection of tasks that were performed for a patient, and be indicative of medical care processes for a medical facility. The patient may belong to a category or have medical characteristics in common, and therefore a collection of EMRs for patients of that category may provide for the determination of typical or standard workflows for that category. For example, patients may be admitted to a medical entity, and determined to have pneumonia. Pneumonia may be a category for which a workflow is determined using the EMRs for patients of the medical entity that have pneumonia.

In an embodiment, a data miner may include components for extracting information from a collection of computerized patient records (CPRs) from an EMR system. The data miner may also be configured to combine all of the evidence in a principled fashion over time, drawing inferences from the combination process. The inferences may be determined using graphical modeling and/or machine learning techniques. The inferences or relationships between medical tasks shown in the collection of EMRs can be used to determine workflows for a medical entity.

Information may be extracted from any electronic source as well as EMRs. In an embodiment, information may be extracted from any electronic records relating to patients and resources of a medical entity. For example, machine logs and/or medical practitioner schedules may provide a source of information. Upon mining information, characteristics of tasks at the medical entity may be determined. For example, a task may include the characteristics of medical equipment used, medical practitioner performing task, location of task performance, time of task performance, cost of total task performance, or any other characteristic relating to the performance of the task. The typical sequence of tasks may also be determined from extracted information, particularly task sequences for workflows can be determined through an analysis of EMRs. In an embodiment, multiple EMRs from patients with the same condition may indicate a typical order of tasks performed on a patient with the condition. For example, a collection of EMRs of patients with knee injuries may indicate that a typical order of tasks performed includes a physician physical exam, then an X-Ray, then a Magnetic Resonance Image, then a Radiologist reviews the images, then a physician performs a second physical exam. The tasks may be indicated with time/date stamps in the EMRs to show the temporal sequence of tasks. These EMRs may be analyzed, the sequence of tasks aligned and grouped, and a typical sequence, or workflow, may be determined to a statistical certainty. In an embodiment, a sequence of tasks may also be determined by applying a clinical standard sequence to determined medical tasks.

A workflow of a medical entity may be determined by analysis of the EMRs for that medical entity. Characteristics of any task in a workflow, whether developed from EMRs or from another source, may be automatically identified by analysis of EMRs to determine any anomalies leading to better or worse results for the workflow.

Workflows may also be graded based on a result. For example, workflows may be graded or evaluated based on patient outcome criteria or financial criteria. Alternate workflows may be recommended based on the evaluations.

Outliers and/or anomalous tasks may also be determined. This may be done by comparing the sequence, collection, or individual characteristics of tasks to standards. The standards may either be developed through a workflow determination analysis, accepted as a standard in the field by a medical body or organization, or manually constructed and put into place by a medical entity.

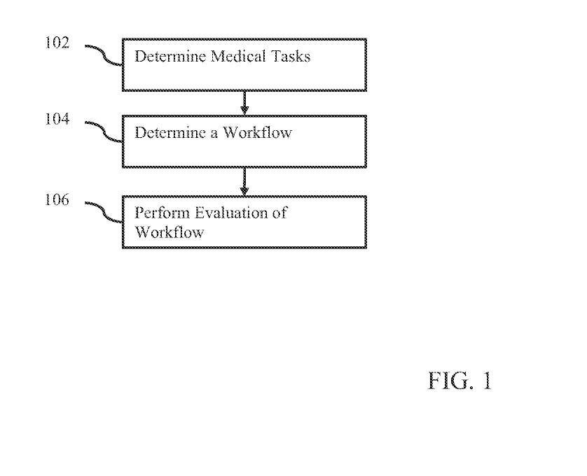

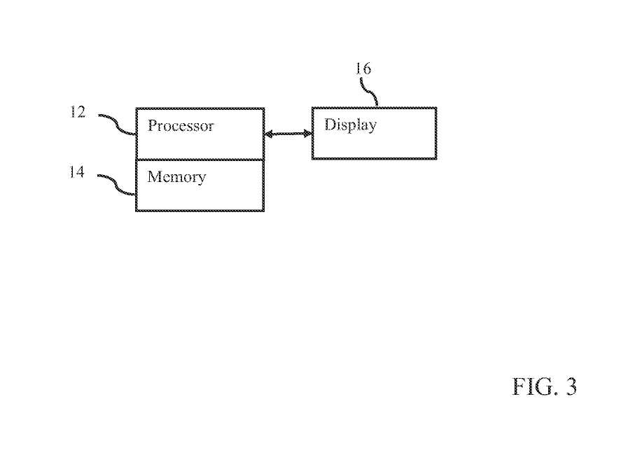

FIG. 1 shows a flow chart diagram of an embodiment of a method for medical entity workflow evaluation. The method is implemented by a computerized physician order entry (CPOE) system, an automated workflow system, a review station, a workstation, a computer, a picture archiving and communication system (PACS) station, a server, combinations thereof, or other system in a medical facility. For example, the system or computer readable media shown in FIG. 3 implements the method, but other systems may be used. Additional, different, or fewer acts may be performed. For example, an act for optimizing performance of a task of a workflow is provided. The method is implemented in the order shown or a different order. For example, acts 102, 104, and 106 may be performed in parallel.

In act 102, a plurality of medical tasks is determined based on an analysis of a plurality of EMRs and/or other electronic records. As described above, EMRs contain data relating to patients and the medical procedures performed on the patients while in the care of a medical facility. This EMR information provides a timeline and model of a patient's experience in the medical facility. Individual records of the procedures in an EMR may indicate specific medical tasks executed by a medical entity with respect to the patient. For example, an X-Ray image may have associated information that indicates the task of performing the X-Ray procedure on the patient while being in the care of the medical facility.

The historical medical data of a medical entity contained in a collection of EMRs may be mined to determine tasks and task characteristics. Any data mining may be used, such as is disclosed in U.S. Pat. No. 7,617,078, the disclosure of which is incorporated herein by reference.

Tasks and task characteristics to be identified may be determined by any method. In an embodiment, known clinical standards and procedural criteria are used. In an embodiment, tasks and task characteristics are learned through a machine learned model. For example, a machine learning model may be provided EMRs of known members of a medical category from an EMR database of a medical entity. The machine learned model may then analyze the known EMRs to determine common or relative tasks and/or task characteristics among the EMRs. The tasks or combination of tasks most common or occurring with a predetermined frequency in the EMRs of the patients may be identified as belonging in a workflow. More than one workflow and corresponding tasks may be identified for a given category. For example, the tasks for two or more alternative workflows for patients in a same category are identified.

Task characteristics may involve any information relating to the task saved in an electronic record. Task characteristics may be stored as fields in an ECR. For example, an X-Ray electronic record may indicate the medical practitioner that performed the X-Ray procedure, a specific X-Ray machine used to perform the procedure, the time the procedure was performed, the type of image obtained, the part of the patient's body imaged, or any other information related to the performance of the task. Task characteristics may also be stored in electronic medical equipment logs, electronic medical practitioner logs, electronic facility janitorial logs, or any other source of information relating to a task. For example, an electronic medical equipment log may be coupled to an EMR by a common field indicating a specific machine. Using this coupled relationship, the information in the electronic medical equipment log may be correlated to a specific piece of medical equipment used in a task. Further characteristics may then be determined for the task such as amount of time since last medical equipment calibration, number of procedures performed on medical equipment since last maintenance, specific parts included in medical machine during the task, or any other characteristic of the medical equipment stored in the log relating to a performed task.

In an embodiment, an analysis may involve determining characteristics for medical tasks of a medical entity. Tasks may have information associated with the task that characterizes the task. This information may be contained directly in an EMR, or in an associated record such as an equipment log or medical practitioner schedule. The characteristics may be any characteristic for which data exists relating to a task. The information may include the medical equipment used, information related to the medical equipment used, medical practitioner performing task, location of task performance, time of day task performance, cost of total task performance, or any other characteristic relating to the performance of the task.

In an embodiment, a characteristic may be a cost of the task which represents the total cost to a medical entity or patient for performing the task. Involved in a task cost may be salaries or costs of medical practitioners, the costs associated with disposable equipment such as bandages used in the task, a cost assigned to the use of a location in the medical entity, a cost associated with the time of use of equipment, or any other cost attributed to the performance of the task.

In an embodiment, a characteristic may be a required sequential dependency of a task. The characteristic may be that a particular task is dependent on the performance of another task. This dependence may be a medical requirement, or a best practice procedure for a medical entity. For example, an admittance task may be required prior to a formal physician examination task to determine basic health information of the patient, such as height, weight, blood pressure, and body temperature. This dependency characteristic may be determined through the analysis of the EMRs by determining that in a predominant number of EMRs, an admittance task is performed immediately prior to a physician formal examination task. The admittance task may not be performed prior to a physician formal examination task in 100% of the records, but a statistical analysis may provide adequate assurance of an existence of the dependency.

In act 104, a workflow of the medical entity is determined based on a sequence of medical tasks, the sequence determined based on the analysis of the plurality of electronic medical records. The procedures recorded in an EMR may be extracted and determined to be tasks that when performed in a sequence are a workflow. A sequence may be determined by using date or time data associated with a task, and ordering tasks by the associated time data. In an embodiment, the data or time data may be considered a characteristic of the task.

In an embodiment, workflows may be determined by applying machine learned models to EMRs or other data relating to tasks or task characteristics. Any machine learned model capable of determining workflows may be used.

A determined workflow may be a particular workflow for a patient, or a determined workflow for patients with particular types or characterizations of conditions. Certain EMRs may be characterized similarly. For example, the EMRs of patients with similar conditions, such as pneumonia, may be characterized together. An analysis may be performed on these characterized EMRs to determine common lists of tasks, and task sequences, to determine a workflow for patients with pneumonia for the medical facility. Similar analysis may be performed to determine other types of determined workflows for a medical entity. The workflow may be for diagnosis, treatment, or both. The workflow may be for all or part of the diagnosis or treatment (e.g., workflow for a department).

A medical workflow may be determined from electronic medical equipment or machine logs, electronic medical practitioner logs, electronic facility janitorial logs, or any other source of information relating to a task. A medical workflow may be determined from any of the sources of information individually, or in combination with other sources of information. In an embodiment, a medical workflow may be determined through an analysis of medical equipment logs, without an analysis of other sources of information. For example, a machine log for an X-Ray machine may store information or characteristics relating to medical procedures performed using the X-Ray machine. The information may be stored with temporal indicators or time stamps. For example, a series of acts that are determined to comprise a procedure may include:

08/09/13 12:04:10 Table Rotated 35 degree clockwise
08/09/13 12:05:30 Table Retracted
08/09/13 12:07:55 Scan Started
08/09/13 12:09:15 Scan Completed

The acts may be considered tasks, and thus the combination of acts may in itself comprise a workflow for the specific X-Ray machine. The combination of acts may also be a task in a workflow comprising multiple tasks. In an embodiment, multiple machine logs of multiple pieces of medical equipment may be analyzed together to determine medical workflows. For example, a patient identifier may be stored with each act, and the patient identifiers may be tracked across machine logs to determine a temporal order of acts and/or tasks performed across multiple pieces of medical equipment. A medical workflow may be determined from system level accumulation of machine logs. Multiple acts from multiple machine logs may be tied using a common element, such as a patient number. For example, a series of recorded acts from a system may include:

08/09/13 12:04:10 Patient 1011 MRI Ordered
Source: order entry from workstation
08/09/13 12:05:30 Patient 1011 Arrives at Floor 3
Source: patient wrist band scan at floor 3
08/09/13 12:07:55 Patient 1011 Arrives at Radiology Lab
Source: patient wrist band scan at lab

Other common elements may be used as well, such as a physician identifier, or other data.

A medical workflow may be determined from other medical facility systems as well. For example, a medical entity financial system may provide information relating to tasks such as billing codes and other information in electronic billing records. In an embodiment, a medical workflow may be determined solely from an analysis of medical entity financial records. For example, electronic billing records for a patient may be mined based on common patient numbers. The billing codes associated with the records, as well as recorded dates associated with the performance of the procedure corresponding to the billing code, may indicate a medical workflow.

In act 106, an evaluation of the workflow is performed based on a predefined criterion. The predefined criterion may be any criteria used by a medical entity to assess the quality or effectiveness of a workflow. Predefined criteria may be financial, patient outcome oriented, procedural standard oriented, or any other predefined criteria.

In an embodiment, financial outcomes may be used as predefined criteria. For example, a certain cost may be determined to be an effective cost for a medical entity for a particular category or type of workflow. The cost of a determined workflow may be determined using cost characteristics of each of the tasks in the determined workflow. The total cost of the determined workflow may be compared to the effective cost to determine adequacy of the workflow from a fiscal standpoint for the medical entity.

In an embodiment, patient outcomes may be used as predefined criteria. For example, a certain patient outcome may be determined by a medical entity. Patient outcomes may be as simple as “positive” or “negative” based on the experience and result of a patient's treatment. Patient outcomes may also be more delineated such as “Full Recovery”, “Partial Recovery”, or “Relapse”. Determined workflows may be evaluated to fit with the particular patient outcome. Certain outcomes may be determined by a medical entity as successful, such as “Full Recovery”. Certain outcomes may be determined by a medical entity as unsuccessful, or negative, such as “Relapse”.

In an embodiment, a predefined workflow of a medical entity may be used as predefined criteria. For example, a workflow may be designed by a medical entity for patients having a condition. The determined workflow may be compared to the designed workflow to determine deviations from the designed workflow. In an embodiment, some tasks in the designed workflow may be determined more important than other tasks, and only important tasks are compared. In an embodiment, important tasks are weighted heavier in a comparison score, and a threshold comparison score is used to evaluate whether the determined workflow is acceptable. A comparison score not meeting the threshold would be considered a negative score. In an embodiment, the time between tasks is taken into account, and requirements for time between tasks are also included in an evaluation of a determined medical workflow.

The evaluation criteria may be used as part of the machine learning to determine tasks performed in act 102 and/or to determine the workflow in act 104. The criterion or criteria are used to stratify or cluster EMRs for different patients to find a workflow and/or characteristics of workflow tasks that provide optimized outcome, cost, or other measure of quality or effectiveness.

A negative medical task of the identified medical tasks of a determined workflow may be identified as negatively contributing to the evaluation. For example, for predefined cost criteria, a task, or the tasks, involving the highest cost of a determined workflow are identified. In another example, a negative patient outcome for a determined workflow may be grouped, and similar task characteristics may be identified. The characteristic may be medical practitioner performing the task, medical equipment used in the task, location of task, time of performance of task, or any characteristic identified as being statistically consistent with the negative patient outcomes for a determined workflow. In another example, specific task deviations from a designed workflow may be identified.

In an embodiment, all of the tasks of a determined workflow are ranked based on a measuring characteristic used in the predefined criterion. For example, when workflow cost is a predefined criterion, all tasks may be ranked by cost, and the highest cost tasks may be identified.

An identified negative task may be compared with other medical tasks of a same category to identify an inconsistency, or other identifying characteristic, from the other medical tasks. For example, an X-Ray task may be compared to other X-Ray tasks to determine a characteristic that is different from the other X-Ray tasks. The characteristic may be a length of time for the X-Ray task, or even a cost of an X-Ray task when compared to other X-Ray tasks. This identified inconsistency or characteristic may be an indicator of a negative influence on an evaluation of a workflow.

An alternate workflow may be recommended. In an embodiment, an alternate workflow recommendation may be based on an identified negative task. The alternate workflow may be constructed to minimize the contribution of the negative task to the evaluation of the workflow.

In an embodiment, recommending an altered workflow may involve identifying a category for a medical task negatively contributing to an evaluation of a workflow. Determining an alternate medical task of the same category that negatively contributes to the evaluation less than the negative medical task, and replacing the negative medical task with the alternate medical task. For example, an X-Ray task identified as a negative task may have an associated cost characteristic. Another X-Ray task, using a different X-Ray machine, or different medical practitioners, may have a lower cost characteristic. A recommended alternate workflow may involve replacing the determined X-Ray task, with the alternate X-ray task having a lower cost such that total cost of the workflow may be lower.

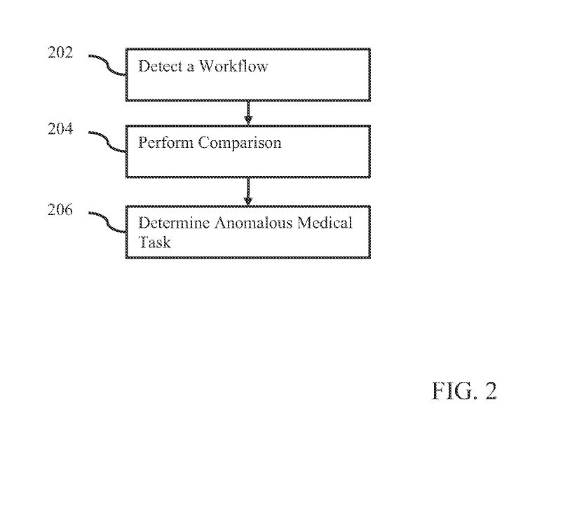

FIG. 2 shows a flow chart diagram of an embodiment of a method for evaluating workflows comprising medical tasks of a medical entity. The method is implemented by a computerized physician order entry (CPOE) system, an automated workflow system, a review station, a workstation, a computer, a picture archiving and communication system (PACS) station, a server, combinations thereof, or other system in a medical facility. For example, the system or computer readable media shown in FIG. 3 implements the method, but other systems may be used. Additional, different, or fewer acts may be performed. The method is implemented in the order shown or a different order. For example, acts 202, 204, and 206 may be performed in parallel.

In act 202, a workflow is detected from an electronic medical record for a patient of a medical entity. The workflow may be a workflow for a specific condition of the patient. The EMR of a patient may also indicate multiple workflows for multiple conditions of the same patient.

The workflow may be detected using any method. The method of determination may identify tasks and sequences of tasks that indicate the existence of a known workflow. A recorded condition of a patient may also identify the existence of a workflow. In an embodiment, a machine learned model is applied to an EMR of a patient to determine a workflow. For example, the workflow is created as discussed above for FIG. 1. Other sources, such as a manually created workflow, may be used.

In act 204, a comparison is performed of the workflow to an established workflow. The established workflow may be a workflow designed by a medical entity and established as a standard of care for a condition. The established workflow may also be a workflow determined from an analysis of a plurality of EMRs for patients of a medical entity. For example, workflows automatically created from EMRs of different medical entities are compared.

The comparison of act 204 may be performed using any method. The workflow being compared may be for a patient with the same, or similar, condition as a condition determined for an established workflow. The workflow and the established workflow may be aligned such that categories of tasks are identified, and characteristics of the tasks are aligned. In an embodiment, the established workflow indicates task categories, and the tasks of workflow are aligned with the task categories in chronological order. Tasks may also be aligned sequentially in chronological order of tasks performed in the workflow and tasks as indicated in the established workflow. In an embodiment, graphical methods may be used to compare a determined workflow to an established workflow. In another embodiment, quantifications of characteristics are grouped and analyzed using statistical methods.

In act 206, an anomalous medical task of the workflow is determined based on the comparison. The anomalous task may be a task having a characteristic that is a statistical outlier among characteristics of similar tasks.

In an embodiment, an anomalous task may be a task performed not in an order indicated in the established workflow. For example, a determined workflow may indicate that a patient is being sent to a surgery task, prior to the completion of an imaging task, such as an X-Ray, that the established workflow indicates is performed prior to the surgery task. In an embodiment, an alarm may be initiated upon the detection of an anomalous medical task.

Extra tasks or failure to perform a task may be identified as an anomalous task. In an embodiment, an alignment of workflow tasks and established workflow tasks may indicate that more tasks are included in the workflow than are indicated in the established workflow. To identify the specific extra task, categories may be established for the tasks, and it may be identified that each task category of the established workflow has a slotted number of tasks and corresponding tasks in the workflow being compared have filled the available slots. Any extra tasks, not filling a slot, may be considered an anomalous task. Also, if there is a slot left open in the established workflow, it may indicate an anomalous, or missing, task. In an embodiment, multiple anomalous tasks may be determined. For example, there may be multiple open slots and multiple leftover workflow tasks not fitting an available slot

Tasks in categories may be compared using category characteristics to find anomalies. Also, an established workflow may include established categories for tasks that have characteristic values determined to be normal for the tasks in the category. In this way, a category for an anomalous task may be identified and an anomaly or anomalous characteristic of the anomalous medical task may be determined based on the medical tasks for the category. For example, an average cost of an X-Ray task may be $2,100. The X-Ray task cost of a determined workflow may be determined to be $3,700. The $3,700 X-Ray task cost may be determined to be an anomaly, or anomalous characteristic, and consequently the task may be considered an anomalous task.

FIG. 3 shows a system for medical entity workflow evaluation. The system is a server, network, workstation, computer, database, or combinations thereof. The system 10 includes a processor 12, a memory 14, and a display 16. Additional, different, or fewer components may be provided. For example, the system includes a scanner, a network connection, a wireless transceiver or other device for receiving patient information and/or communicating patient information to other systems. A wireless transceiver may allow for communication with a physician's mobile device for displaying information such as an alarm indicating an out of sequence anomalous task. Preferred task characteristics may also be included in the established workflow, and the characteristics of the tasks in the workflow may be aligned with the preferred task characteristics.

The memory 14 is a buffer, cache, RAM, removable media, hard drive, magnetic, optical, database, or other now known or later developed memory. The memory 14 is a single device or group of two or more devices. The memory 14 is shown within the system, but may be outside or remote from other components of the system, such as a database or PACS memory.

The memory 14 stores an EMR for a patient. Multiple EMRs of other patients may also be stored on the memory 14. In an embodiment, the memory 14 is operable to store a plurality of electronic medical records of a plurality of patients of a medical entity as well as a specific electronic medical record of the patient.

The memory 14 is additionally or alternatively a non-transitory computer readable storage medium with processing instructions. The memory 14 stores data representing instructions executable by the programmed processor 12 for determining a medical category for a patient. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 12 is a server, general processor, digital signal processor, graphics processing unit, application specific integrated circuit, field programmable gate array, digital circuit, analog circuit, combinations thereof, or other now known or later developed device for medical category determination. The processor 12 is a single device, a plurality of devices, or a network. For more than one device, parallel or sequential division of processing may be used. Different devices making up the processor 12 may perform different functions, such as a handwriting detector by one device and a separate device for communicating or processing the detected handwritten data. In one embodiment, the processor 12 is a control processor or other processor of a computerized data entry system for an EMR storage or database system. The processor 12 operates pursuant to stored instructions to perform various acts described herein.

The processor 12 is configured by software or hardware to determining and evaluating workflows. The processor 12 may be configured to determine a plurality of medical tasks based on an analysis of a plurality of electronic medical records of a medical entity stored on the memory 14. The processor 12 may be further configured to determine a workflow of the medical entity based on a sequence of the medical tasks, the sequence determined based on the analysis of the plurality of electronic medical records, and perform an evaluation of the workflow based on predefined criteria.

The display 16 is a CRT, LCD, plasma, projector, printer, or other output device for showing an image. The display 16 displays a user interface with an image. The display may also be configured to display a graphical representation of workflows, tasks, and task characteristics determined from EMRs. The user interface may also be for the entry of information, such as information that may be characteristics that indicate the inclusion of a patient in a medical category. The user interface may be for entering information into an EMR. The user interface may also display an evaluation of a determined workflow

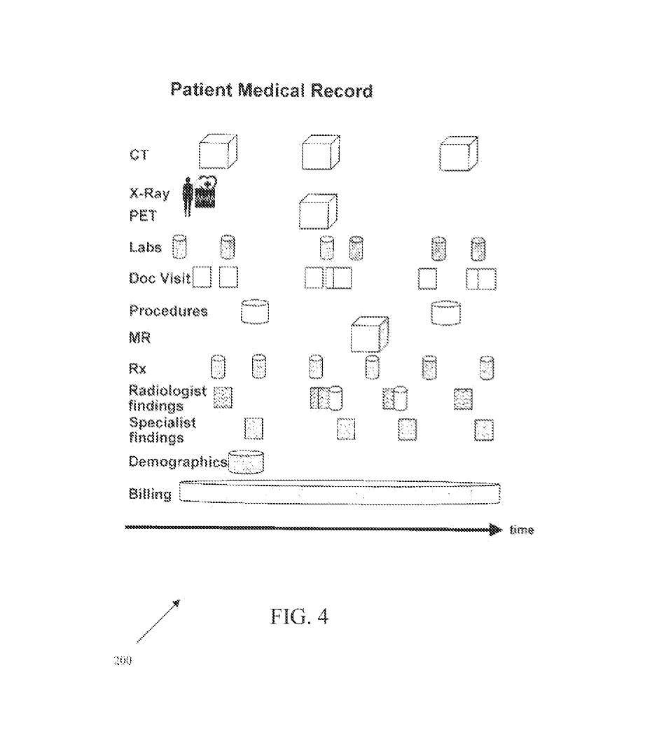

FIG. 4 shows an exemplary EMR 200. Health care providers may employ automated techniques for information storage and retrieval. The use of an EMR to maintain patient information is one such example. As shown in FIG. 4, an exemplary EMR 200 includes information collected over the course of a patient's treatment or use of an institution. This information may include, for example, computed tomography (CT) images, X-ray images, laboratory test results, doctor progress notes, details about medical procedures, prescription drug information, radiological reports, other specialist reports, demographic information, family history, patient information, and billing(financial) information. Any of this information may provide for a workflow, task, or task characteristic determination.

An EMR may include a plurality of data sources, each of which typically reflects a different aspect of a patient's care. Alternatively, the EMR is integrated into one data source. Structured data sources, such as financial, laboratory, and pharmacy databases, generally maintain patient information in database tables. Information may also be stored in unstructured data sources, such as, for example, free text, images, and waveforms. Often, characteristics, such as key clinical findings, are stored within unstructured physician reports, annotations on images or other unstructured data source.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for medical entity workflow evaluation, the method comprising:
determining, with a processor, a plurality of medical tasks based on an analysis of a plurality of electronic medical of a medical entity;
determining, with the processor, a workflow of the medical entity based on a sequence of the medical tasks; and
performing, with the processor, an evaluation of the workflow based on a predefined criterion.

2. The method of claim 1, wherein the analysis further comprises determining characteristics for the medical tasks.

3. The method of claim 2, wherein the characteristics comprise a cost of a task or a task time to completion.

4. The method of claim 2, wherein the characteristics comprise a dependency of a first medical task of the medical tasks on a different medical task of the medical tasks.

5. The method of claim 2, wherein the characteristics comprise a medical practitioner or a time of day.

6. The method of claim 1, wherein the analysis comprises the application of a machine learned model to the plurality of electronic records.

7. The method of claim 1, wherein the predefined criterion comprise a financial outcome or a patient outcome.

8. The method of claim 1, wherein the predefined criterion comprise a workflow for the medical entity.

9. The method of claim 1 further comprising identifying a negative medical task of the medical tasks, the negative medical task negatively contributing to the evaluation.

10. The method of claim 9, wherein identifying a negative task comprises ranking all the medical tasks of the workflow by the predefined criterion.

11. The method of claim 9 further comprising identifying an inconsistency of the negative task with tasks of a same category.

12. The method of claim 9 further comprising recommending an altered workflow minimizing the contribution of the negative task.

13. The method of claim 12, wherein recommending an altered workflow comprises:
identifying a category for the medical task negatively contributing to the evaluation;
determining an alternate medical task of the category that negatively contributes to the evaluation less than the negative medical task; and
replacing the negative medical task with the alternate medical task.

14. A non-transitory computer readable storage medium having stored therein data representing instructions executable by a programmed processor for evaluating workflows comprising medical tasks of a medical entity, the storage medium comprising instructions for:
detecting a workflow from an electronic medical record for a patient of a medical entity;
performing a comparison of the workflow to an established workflow; and
determining an anomalous medical task of the workflow based on the comparison.

15. The medium of claim 14, wherein detecting a workflow comprises applying a machine learned model to an electronic medical record of a patient.

16. The medium of claim 14, wherein the established workflow is derived from the application of a machine learned model to a plurality of medical records for patients of a medical entity.

17. The medium of claim 14, wherein the established workflow is comprised of a sequence of medical tasks, and determining the anomalous medical task comprises the detection of an out of sequence medical task of the medical tasks.

18. The medium of claim 14, wherein determining an anomalous medical task comprises:
identifying a category for the anomalous medical task; and
determining an anomaly of the anomalous medical task based on medical tasks for the category.

19. The medium of claim 14, wherein the instructions are further executable to initiate an alarm upon the detection of the anomalous medical task.

20. A system for medical entity workflow evaluation, the system comprising:
at least one memory operable to store electronic records relating to patients and resources of a medical entity; and
a first processor configured to:
determine a plurality of medical tasks based on an analysis of the electronic records of the medical entity;
determine a workflow of the medical entity based on a sequence of the medical tasks; and
perform an evaluation of the workflow based on predefined criteria.

21. The system of claim 20, wherein the analysis comprises the application of a machine learned model to the electronic records.

22. The system of claim 20, wherein the predefined criteria comprise a financial outcome or a patient outcome.

23. The system of claim 20, wherein the predefined criteria comprise another workflow for the medical entity.

24. The system of claim 20, wherein the sequence is determined based on the analysis of the electronic records.

25. The system of claim 20, wherein the electronic records comprise electronic records selected from the group consisting of machine logs, medical practitioner schedules, patient electronic medical records, or a combination thereof.
